(19) United States
(12) Patent Application Publication
(10) Pub. No.: US 2018/0078668 A1
(43) Pub. Date: Mar. 22, 2018

(54) LAMP STRUCTURE

(71) Applicants: CHIAO-AN HSIAO, Taoyuan (TW); YU-HSIU LU, Taoyuan (TW); CHUN-HUI WU, Taoyuan (TW)

(72) Inventors: CHIAO-AN HSIAO, Taoyuan (TW); YU-HSIU LU, Taoyuan (TW); CHUN-HUI WU, Taoyuan (TW)

(21) Appl. No.: 15/270,168

(22) Filed: Sep. 20, 2016

(51) Int. Cl. A61L 9/20; F21V 15/01; F21S 9/02; F21V 8/00; F21V 7/00

(52) U.S. Cl. CPC A61L 9/205; F21V 15/01; F21S 9/02; F21Y 2115/10; G02B 6/0055; F21V 7/0025; G02B 6/0051

(57) ABSTRACT

A lamp structure includes a casing, a circulation module, a power supply module, a light emitting module, an air vent, and a filter. A nano antiviral material composed of titanium dioxide and nanosilver is coated onto the filter installed at a lower end portion of the lamp structure, and the air circulation device is provided for obtaining fresh clean air after the air is filtered by the nano antiviral material and the filter, so as to achieve the effects of purifying air, deodorizing, resisting viruses and bacteria, and removing pollutants from the environment effectively for the area irradiated by the light of the lamp.

### FIELD OF THE INVENTION

The present invention relates to a lamp structure, more particularly to the lamp structure having an air circulation device and a filter coated with a nano antiviral material.

### BACKGROUND OF THE INVENTION

Due to global climate anomalies, pollutions resulted from over-exploitation of land, too heavy traffic, and too much construction, the quality of our living environment becomes very poor. Since people usually stay in a closed air-conditioned place to work in daytime or on street of a city fully exposed to waste gases, we always want to have a natural clean, quiet and comfortable environment to rest at nighttime. In addition, air is a necessary element of life, and the quality of air directly reflects the quality of the living environment, and a lamp structure is an important tool for providing a light source indoors and at nighttime and bringing the convenience of providing the required light for the surrounding environment.

With reference to FIG. 1 for a perspective view of a conventional lamp structure, the conventional lamp structure formed by combining a lamp base (P1), a lamp housing (P2), and a cover plate (P3), wherein the lamp housing (P2) and the cover plate (P3) are provided for protecting the lamp base (P1) with a built-in light source. The conventional lamp structure just provides the illumination function. Although the conventional lamp structure brings the convenience of lighting the surrounding environment, yet the quality of air is worsening, and floating bacteria, fungi, or microorganisms are everywhere, and the conventional lamp structure does not have the air purification function. In a closed circulated air conditioning environment, the accumulation of waste gases for a long time and viruses and bacteria carried by personnel passing in and out are major factors to the deterioration of the air quality and a cause to the discomfort of human body. Therefore, it is an important subject for related manufacturers to design an innovative lamp structure and combine the illumination and air circulation functions to provide the air purifying, deodorizing, anti-virus, antibacterial functions for the space where the lamp is situated.

### SUMMARY OF THE INVENTION

In view of the aforementioned problems, the inventor of the present invention based on years of experience of the related industry to conduct extensively researches and experiments, and finally developed a lamp structure. Therefore, it is a primary objective of the present invention to provide a lamp structure comprising an air circulation device and a filter coated with a nano antiviral material, wherein the nano antiviral material mainly composed of titanium dioxide and nanosilver is coated onto the filter of the lamp structure, and then fresh clean air can be obtained after the air circulation device allows the air to be filtered by the nano antiviral material and the filter, so as to achieve the air purifying, deodorizing, resisting viruses and bacteria, removing pollutants from the environment for the area irradiated by the light of the lamp.

To achieve the aforementioned and other objectives, the present invention discloses a lamp structure comprising a casing, a circulation module, a power supply module, a light emitting module, an air vent, and a filter, wherein the casing comprises an upper casing and a bottom cover, the upper casing and the bottom cover jointly form an accommodating space; the circulation module is installed in the accommodating space, and the circulation module includes a motor and a fan coupled to the motor to achieve the air circulation effect, wherein the power supply module is installed in the accommodating space and electrically coupled to the circulation module for supplying electric power to the motor, and the motor drives the fan to rotate for air circulation; the light emitting module is installed in the accommodating space and electrically coupled to the power supply module, so that the light emitting module emits light by the electric power of the power supply module; the air vent is installed at an outer end portion of the accommodating space, so that air may enter or flow out through the air vent to improve the air circulation; the filter is installed inside or outside the air vent or clamped at the air vent, and a nano antiviral coating is coated onto the interior or the outer surface of the filter, such that after air passes through the nano antiviral coating, a fresh clean air is obtained, and the invention has the effects of purifying air, deodorizing, resisting viruses and bacteria, and removing pollutants from the environment for the area irradiated by the light of the lamp.

According to the aforementioned technical characteristics, the nano antiviral coating can purify air, and the circulation module can accelerate the air circulation, and both of these functions can achieve the synergic effect of accelerating the air purification.

According to the aforementioned technical characteristics, a top cover is further installed between the upper casing and the bottom cover, and the top cover and the upper casing jointly form a first accommodating space, and the top cover can reflect the light emitted by the light emitting module, so that the light reflected by the top cover may be outputted in a predetermined direction towards the lamp structure to enhance the light output effect.

According to the aforementioned technical characteristics, the light emitting module is coupled to a cooling aluminum plate, and the cooling aluminum plate is capable of improving the heat dissipation effect of the light emitting module, and the heat dissipation can be accelerated by rotating the fan of the circulation module to drive the air circulation.

According to the aforementioned technical characteristics, the air vent includes an air inlet and an air outlet, and the synergic effect of accelerating the air purification can be achieved by enhancing the convection according to the principle of fluid dynamics.

According to the aforementioned technical characteristics, the air inlet or the air outlet is installed at an outer end portion of the accommodating space.

According to the aforementioned technical characteristics, the filter is installed at the air inlet or the air outlet, or installed separately at the air inlet and the air outlet.

According to the aforementioned technical characteristics, the filter is installed inside or outside the air inlet or clamped at the air inlet.

According to the aforementioned technical characteristics, the filter is installed inside or outside the air outlet or clamped at the air outlet.

According to the aforementioned technical characteristics, the nano antiviral coating includes titanium dioxide and nanosilver.

According to the aforementioned technical characteristics, the accommodating space has a backlight module installed therein.

According to the aforementioned technical characteristics, the backlight module is disposed between the light emitting module and the bottom cover.

According to the aforementioned technical characteristics, the backlight module includes a reflective plate, a light guide plate, and a light diffuser plate.

According to the aforementioned technical characteristics, the nano antiviral coating is coated onto the outer surface of the light diffuser plate.

According to the aforementioned technical characteristics, the power supply module is a battery.

According to the aforementioned technical characteristics, the light emitting module is a light emitting diode (LED).

According to the aforementioned technical characteristics, an ultraviolet light emitting diode is further installed at the air inlet for enhancing the disinfection effect and achieving the synergic effect of accelerating the air purification.

In the lamp structure of the present invention, a nano antiviral material composed of titanium dioxide and nanosilver is coated onto the filter of the lamp structure, and an air circulation device is provided for obtaining fresh clean air after the air is filtered by the nano antiviral material and the filter, so that the present invention has the advantages of purifying air, deodorizing, resisting viruses and bacteria, and removing pollutants from the environment effectively for the area irradiated by the light of the lamp.

In addition, the lamp structure of the present invention includes a backlight module installed therein for improving the uniformity of the light emitting brightness, and a nano antiviral material composed of titanium dioxide and nanosilver is coated onto the outer surface of a light diffuser plate of the backlight module, such that after the light emitted by the light emitting module passes through the light diffuser plate of the backlight module, the effects of purifying air, deodorizing, resisting viruses and bacteria, and removing pollutants from the environment for the area irradiated by the light of the lamp can be achieved effectively.

### BRIEF DESCRIPTION OF THE DRAWINGS

FIG. 1 is a perspective view of a conventional lamp structure;

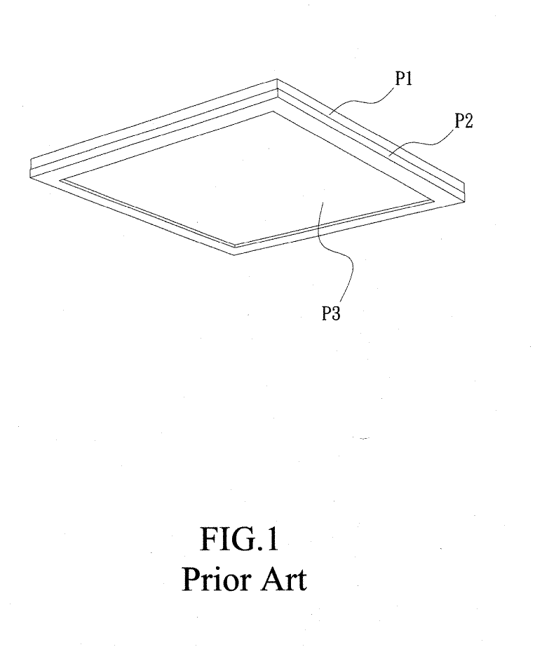

FIG. 2 is a perspective view of a lamp structure in accordance with a first preferred embodiment of the present invention;

FIG. 3 is an exploded view of a lamp structure in accordance with the first preferred embodiment of the present invention;

FIG. 4 is an exploded view of a lamp structure in accordance with a second preferred embodiment of the present invention; and

FIG. 5 is a schematic view of a filter of a lamp structure in accordance with the first preferred embodiment of the present invention.

### DESCRIPTION OF THE PREFERRED EMBODIMENTS

To make it easier for our examiner to understand the objective of the invention, its structure, innovative features, and performance, we use a preferred embodiment together with the attached drawings for the detailed description of the invention. It is noteworthy that all figures are drawn for ease of explanation of the basic teachings of the present invention only; the extensions of the figures with respect to number, position, relationship, and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

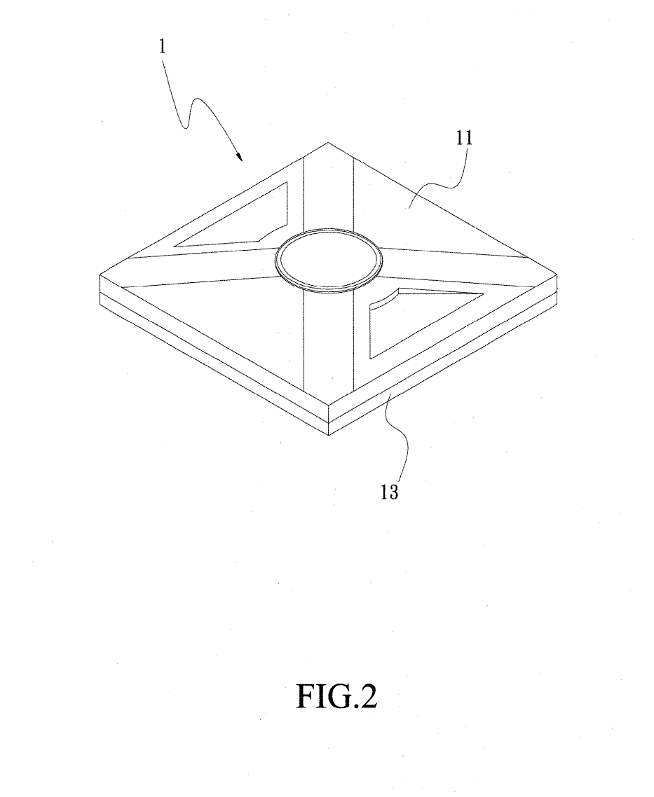

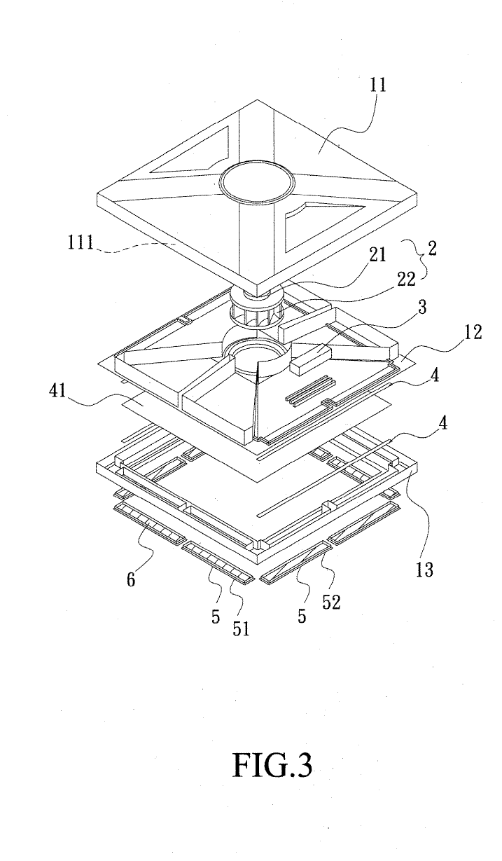

With reference to FIGS. 2-3 and 5 for a perspective view and an exploded view of a lamp structure and a schematic view of a filter in accordance with a preferred embodiment of the present invention, the lamp structure of the present invention comprises the following components:

A casing (1) further comprises an upper casing (11) and a bottom cover (13), and the upper casing (11) and the bottom cover (13) jointly form an accommodating space (not shown in the figures), wherein this embodiment further comprises a top cover (12) installed between the upper casing (11) and the bottom cover (13), and the upper casing (11) is covered onto the top of the top cover (12), and a first accommodating space (not shown in the figures) is formed between the upper casing (11) and the top cover (12), and the top cover (12) is covered onto the top of the bottom cover (13), and a second accommodating space (not shown in the figures) is formed between the top cover (12) and the bottom cover (13); in addition, the casing (1) is substantially in a ring shape such as a square, a rectangular or a circular shape; and the bottom cover (13) is also in a ring shape such as a square, a rectangular or a circular shape; in a preferred embodiment of the present invention, the casing (1) is in a square shape, and in other words, the upper casing (11), the top cover (12), and the bottom cover (13) are also in a square shape, and the first accommodating space is communicated or not communicated with the second accommodating space. For example, a channel or a through hole is provided for flowing air between the first accommodating space and the second accommodating space. The accommodating space includes the first accommodating space and the second accommodating space.

A circulation module (2) is installed in the accommodating space, and the circulation module (2) may be installed in the first accommodating space or the second accommodating space. In this preferred embodiment, the circulation module (2) is installed on a fan holder (111) at a lower end portion of the upper casing (11), and the circulation module (2) includes a motor (21), and a fan (22) coupled to the motor (21), and the motor (21) drives the fan (22) to rotate to produce an airflow and enhance the air circulation. The fan (22) is installed in the first accommodating space or the second accommodating space. In a preferred embodiment, the fan (22) is installed in the second accommodating space.

A power supply module (3) is installed in the accommodating space, wherein the power supply module (3) may be installed inside the first accommodating space and electrically coupled to the circulation module (2) for supplying the required electric power to the motor (21) of the circulation module (2). In addition, the power supply module (3) may be a battery.

At least one light emitting module (4) is installed in the accommodating space. In this embodiment, a plurality of light emitting modules (4) is installed in the second accommodating space, and the light emitting modules (4) are coupled to a cooling aluminum plate (41) for cooling the light emitting module (4), and the cooling aluminum plate (41) may be installed between the plurality of light emitting modules (4), wherein the light emitting modules (4) are electrically coupled to the power supply module (3) for receiving the electric power supplied by the power supply module (3) to emit light for the illumination effect. In addition, the light emitting module (4) is a light emitting diode.

At least one air vent (5) is installed in the accommodating space. In this embodiment, each of the air vents (5) includes at least one air inlet (51) and at least one air outlet (52), and the air inlet (51) or the air outlet (52) is disposed at an outer end portion of the accommodating space or the second accommodating space or the first accommodating space. In other words, the air vent (5) is disposed at an outer end portion in the accommodating space or the second accommodating space or the first accommodating space. For example, the air vent (5) may be combined with the bottom cover (13) and disposed at an outer edge of the bottom cover (13) of the second accommodating space.

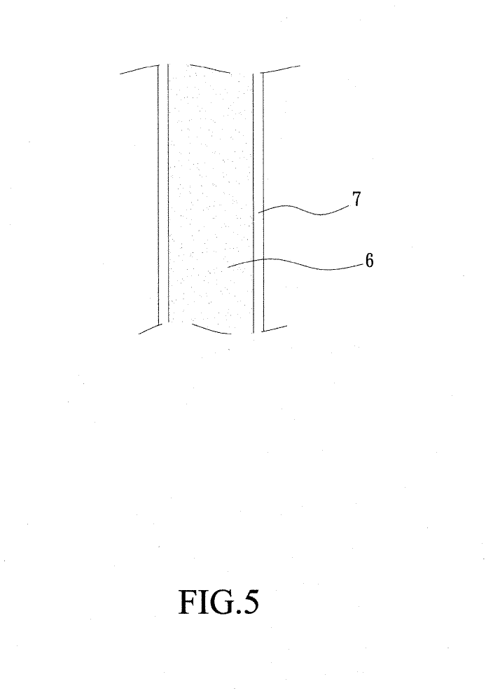

A filter (6) is installed inside or outside the air inlet (51) or clamped at the air inlet (51). In this embodiment, the filter (6) is installed inside the air inlet (51), and a nano antiviral coating (7) is coated onto the interior or the outer surface of the filter (6). In FIG. 5, the nano antiviral coating (7) is coated onto the outer surface of the filter (6), wherein the nano antiviral coating (7) is made of a nano antiviral material, and the nano antiviral material includes titanium dioxide and nanosilver. In other words, the nano antiviral coating (7) includes titanium dioxide and nanosilver. In the embodiment as shown in FIG. 5, the nano antiviral coating (7) is covered onto the outer surface of the filter (6), and the circulation module (2) sucks air into the lamp structure through the air inlet (51) and then discharges the air to the outside through the air outlet (52). Since the outer surface of the filter (6) installed in the air inlet (51) is coated with the nano antiviral coating (7) made of the nano antiviral material, therefore after the sucked air passes through the nano antiviral material, the air becomes purified, so as to achieve the effect of purifying air, deodorizing, resisting viruses and bacteria, and removing pollutants from the environment effectively for the area irradiated by the light of the lamp.

Of course, the filter (6) may also be installed inside or outside the air outlet (52) or clamped at the air outlet (52); or the filter (6) is installed separately at the air inlet (51) and the air outlet (52). In other words, the filter (6) may be installed at the air inlet (51) or the air outlet (52), and the filter (6) may be installed inside or outside the air vent (5) or clamped at the air vent (5).

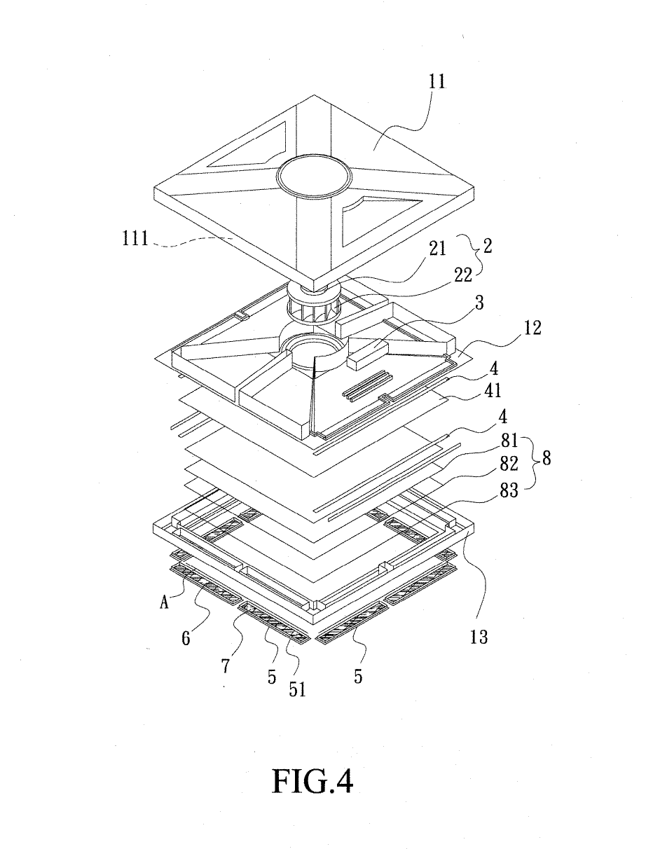

With reference to FIG. 4 for an exploded view of a lamp structure in accordance with the second preferred embodiment of the present invention , the lamp structure has a backlight module (8) installed in the accommodating space, and the backlight module (8) is installed in the first accommodating space or the second accommodating space and disposed between the light emitting module (4) and the bottom cover (13) for improving the uniformity of the light emitting brightness of the lamp structure, and the backlight module (8) includes a reflective plate (81), a light guide plate (82), and a light diffuser plate (83), wherein the outer surface of the light diffuser plate (83) is coated with the nano antiviral coating (7). In the second preferred embodiment of the present invention, the backlight module (8) is installed in the second accommodating space, and the outer surface of the light diffuser plate (83) of the backlight module (8) is coated with the nano antiviral coating (7), so that the light emitted by the light emitting module (4) can pass through the nano antiviral material of the light diffuser plate (83) to achieve the effects of purifying air, deodorizing, resisting viruses and bacteria, and removing pollutants from the environment effectively for the area irradiated by the light of the lamp. In addition, an ultraviolet light emitting diode (A) is installed at the air inlet (51) for the disinfection effect, and the wavelength of the ultraviolet light initiates the photocatalysis of the titanium dioxide and further achieves the effects of purifying air, deodorizing, resisting viruses and bacteria, and removing pollutants from the environment effectively.

To make it easier for our examiner to understand the objective of the invention, its structure, innovative features, and performance, we use a preferred embodiment together with the attached drawings for the detailed description of the invention. It is noteworthy that the embodiments and figures disclosed herein are to be considered illustrative rather than restrictive. When a user wants to select a lamp, the user may select the lamp structure of the present invention capable of purifying air, deodorizing, resisting viruses and bacteria, and removing pollutants from the environment effectively. Firstly, the casing (1) is prepared, wherein the casing (1) is substantially in a square shape and includes the upper casing (11), the top cover (12), and the bottom cover (13) in a ring shape, and the upper casing (11) is covered onto an upper end portion of the top cover (12), and the first accommodating space is formed between the upper casing (11) and the top cover (12), and the top cover (12) is covered onto an upper end portion of the bottom cover (13), and the second accommodating space is formed between the top cover (12) and the bottom cover (13). In addition, the casing (1) is in a square, rectangular or circular shape. The circulation module (2) is installed on the fan holder (111) at the lower end portion of the upper casing (11), wherein the circulation module (2) includes the motor (21), and the fan (22) coupled to the motor (21). In the first accommodating space, the power supply module (3) in form of a battery is installed, wherein the power supply module (3) is electrically coupled to the circulation module (2) for supplying the required electric power to the circulation module (2), and then the plurality of light emitting modules (4) is installed in the second accommodating space, wherein the light emitting modules (4) are stacked in pairs and the cooling aluminum plate (41) is installed in the middle, and the light emitting modules(4) is electrically coupled to the power supply module (3) for receiving the electric power supplied by the power supply module (3) and supplying the electric power for illumination, and then the air vents (5) having the air inlet (51), and the air outlet (52) are installed at the outer end portion of the second accommodating space, wherein the air inlet (51) and the air outlet (52) are arranged adjacent to each other, and finally the filter (6) is installed in the air inlet, and the outer surface of each filter (6) is coated with the nano antiviral coating (7), and the nano antiviral coating (7) is made of a nano antiviral material and the nano antiviral material is composed of titanium dioxide and nanosilver. In the present invention, the nano antiviral material composed of titanium dioxide and nanosilver is coated onto the filter (6) installed at the lower end portion of the lamp structure, and then the circulation module (2) obtains a fresh clean air after the air is filtered by the nano antiviral material and the filter (6), so as to achieve the effects of purifying air, deodorizing, resisting viruses and bacteria, and removing pollutants from the environment effectively for the area irradiated by the light of the lamp.

Compared with the prior art, the present invention has the following advantages:

1. In the lamp structure of the present invention, the nano antiviral material composed of titanium dioxide and nanosilver is coated onto the filter installed at the lower end portion of the lamp structure, so that the air circulation device can obtain a fresh clean air after the air is filtered by the nano antiviral material and the filter, so as to achieve the effects of purifying air, deodorizing, resisting viruses and bacteria, and removing pollutants from the environment effectively for the area irradiated by the light of the lamp.

2. The backlight module is installed in the lamp structure of the present invention for enhancing the light emitting brightness uniformly, and the nano antiviral material composed of titanium dioxide and nanosilver is coated onto the outer surface of the light diffuser plate of the backlight module, such that after the light emitted by the light emitting module passes through the light diffuser plate of the backlight module, the invention achieves the effects of purifying air, deodorizing, resisting viruses and bacteria, and removing pollutants from the environment effectively for the area irradiated by the light of the lamp.

In summation of the description above, the lighting device of the present invention definitely achieve the expected effect as disclosed in the embodiments, and the present invention complies with patent application requirements, and is thus duly filed for patent application.

While the invention has been described by means of specific embodiments, numerous modifications and variations could be made thereto by those skilled in the art without departing from the scope and spirit of the invention set forth in the claims.

What is claimed is:

1. A lamp structure, comprising:
a casing, comprising an upper casing and a bottom cover, and the upper casing and the bottom cover jointly forming an accommodating space;
a circulation module, installed in the accommodating space, and including a motor and a fan coupled to the motor;
a power supply module, installed in the accommodating space, and electrically coupled to the circulation module;
a light emitting module, installed in the accommodating space, and electrically coupled to the power supply module;
an air vent, disposed at an outer end portion of the accommodating space; and
a filter, installed inside or outside the air vent, or clamped at the air vent, and having a nano antiviral coating coated inside the filter or on an outer surface of the filter.

2. The lamp structure of claim 1, further comprising a top cover installed between the upper casing and the bottom cover.

3. The lamp structure of claim 1, wherein the light emitting module is coupled to a cooling aluminum plate.

4. The lamp structure of claim 1, wherein the air vent includes an air inlet and an air outlet.

5. The lamp structure of claim 4, wherein the air inlet or the air outlet is installed at an outer end portion of the accommodating space.

6. The lamp structure of claim 4, wherein the filter is installed at the air inlet or the air outlet, or installed at both of the air inlet and the air outlet.

7. The lamp structure of claim 6, wherein the filter is installed inside or outside the air inlet or clamped at the air inlet.

8. The lamp structure of claim 6, wherein the filter is installed inside or outside the air outlet or clamped at the air outlet.

9. The lamp structure of claim 1, wherein the nano antiviral coating includes titanium dioxide and nanosilver.

10. The lamp structure of claim 1, further comprising a backlight module installed in the accommodating space.

11. The lamp structure of claim 10, wherein the backlight module is disposed between the light emitting module and the bottom cover.

12. The lamp structure of claim 10, wherein the backlight module includes a reflective plate, a light guide plate and a light diffuser plate.

13. The lamp structure of claim 12, wherein the nano antiviral coating is coated onto the outer surface of the light diffuser plate.

14. The lamp structure of claim 13, wherein the nano antiviral coating includes titanium dioxide and nanosilver.

15. The lamp structure of claim 1, wherein the power supply module is a battery.

16. The lamp structure of claim 1, wherein the light emitting module is a light emitting diode.

17. The lamp structure of claim 4, wherein the air inlet further includes a ultraviolet light emitting diode.
